(19) United States
(12) Patent Application Publication
(10) Pub. No.: US 2023/0390244 A1
(43) Pub. Date: Dec. 7, 2023

(54) MODIFIED RELEASE COMPOSITIONS COMPRISING MELATONIN

(71) Applicant: Inventia Healthcare Limited, Mumbai, Maharashtra (IN)

(72) Inventors: RAJAT VISHAL SHAH, Mumbai, Maharashtra (IN); VISHAL JANAK SHAH, Mumbai, Maharashtra (IN); ABDUL SHAJAHAN, Madurai, Tamil Nadu (IN)

(21) Appl. No.: 18/250,782

(22) PCT Filed: Oct. 28, 2021
(86) PCT No.: PCT/IB2021/059955

§ 371 Date: Apr. 27, 2023

(30) Foreign Application Priority Data
Oct. 30, 2020 (IN) 202021047393

(51) Int. Cl. A61K 31/4045; A61K 9/16; A61K 9/00; A61K 9/48; A23G 3/36; A23G 3/40

(52) U.S. Cl. CPC A61K 31/4045; A61K 9/1664; A61K 9/1617; A61K 9/1635; A61K 9/0056; A61K 9/4866; A23G 3/368; A23G 3/40

(57) ABSTRACT

The invention relates to modified release granular composition comprising melatonin and at least one non-swelling release controlling agent. The matrix granular composition as described herein may be specifically comprised of about 1 to 70% by weight of melatonin and about 10 to 70% by weight of non-swelling release controlling agent along with at least one excipient. The invention also relates to a process for preparation, wherein melatonin is uniformly embedded in at least one non-swelling release controlling agent using controlled heat conditions and the matrix granules may be optionally treated with non-swelling polymeric granulating agent to obtain modified release composition. The composition as described herein exhibits about 80% release of melatonin over 6-10 hours. The taste-masked granules can be formulated in suitable oral dosage forms which can be conveniently administered for improvement in quality of sleep to the subjects in need thereof.

The invention relates to modified release compositions comprising melatonin and at least one non-swelling release controlling agent for oral administration. Melatonin composition as described herein may be specifically comprised of about 1 to 70% by weight of melatonin, about 10 to 70% by weight of non-swelling release controlling agent and at least one excipient, which is acceptable in nutraceutical, pharmaceutical and food industry. The composition may be comprised of at least one non-polymeric non-swelling release controlling agent, optionally combined with polymeric non-swelling release controlling agent as part of melatonin granules. The invention also relates to a process for preparation, wherein melatonin is embedded in at least one non-swelling release controlling agent using controlled heat conditions and the matrix granules may be optionally treated with polymeric non-swelling release controlling agent by process of granulation and/or coating to obtain modified release composition. The composition as described herein is effectively taste masked and also exhibits about 80% release of melatonin over 6-10 hours. The taste-masked melatonin granules range in size from 50 to 600 microns and can be formulated in suitable oral dosage forms like gummies, jellies, chewing gums, pellets, minitablets, tablets, capsules, beverages or can be filled in sachets and stick packs for improving quality of sleep in the subjects in the need thereof.

### BACKGROUND

Melatonin, the primary hormone of the pineal gland, plays a very crucial role in maintaining normal circadian rhythms. Endogenous synthesis of melatonin displays a pronounced circadian rhythm. The production of melatonin (N-acetyl-5-methoxytryptamine) from the amino acid tryptophan is primarily nocturnal and is controlled by exposure to cycles of light and dark, independent of sleep. Melatonin synthesized by the pineal gland is released quickly into the bloodstream and then into other bodily fluids, including cerebral spinal fluid (CSF), saliva and bile. Melatonin synthesis is inhibited by exposure to light; production is stimulated during periods of darkness by way of a multi-synaptic neural pathway connecting the pineal gland to the external environment via the retina.

Melatonin has a very short half-life and concentrations are maintained by continual production for 8-10 h during the night. The secretion of this hormone is at peak in the middle of the night and gradually reducing towards the morning. Serum melatonin levels are highest prior to bedtime.

Melatonin deficiency affects the biological clock and results in the disturbance of the physiological functions regulated by the circadian rhythm. There is an age-related decline in the robustness of the circadian clock and melatonin production, thus depriving the brain of an important sleep regulator. Lower production of melatonin was found in patients who suffer from poor sleep quality compared with healthy elderly without such complaint. Older subjects show an increased lag period for the onset of melatonin pulse, and lack of synchronization between melatonin secretion and the middle of the sleep period. This leads to melatonin pulse starts later, peaks later and lower and ends sooner. Thus, the decline in melatonin associated with age (or disease) may act in conjunction with other factors (physical and psychological) to impair sleep at advanced age, thus causing insomnia.

People with insomnia experience higher rates of relationship difficulties, energy deficiency and depressed mood, irritability, tiredness, anxiety, low concentration and poor memory, amongst other health and wellbeing problems. Far from being a minor irritation, insomnia puts sufferers at significantly greater risks of poor mental and physical health ranging from depression, anxiety, immune deficiency and metabolic and cardiovascular disease. Melatonin replacement therapy may replenish the deficiency in the endogenous sleep-regulating hormone, thereby improving sleep quality. Exogenous melatonin simulates/mimics nocturnal circadian physiology in patients with low or abnormal secretion.

Melatonin is also considered to be effective for minimizing the effects of jet lag in travellers crossing multiple time zones. It is also useful to tackle sleep disorders in people working in night shifts by way of its effect in normalizing circadian rhythm, wherein it improves quality and length of daytime sleep. Melatonin can be used for its hypnotic or sedative effect, which helps to induce sleep in certain conditions, where sound sleep is desired for recovery in certain ailments. Numerous neuropsychiatric and neurodevelopmental conditions, including mental retardation, epilepsy, autism, Alzheimer's disease, schizophrenia, and seasonal affective disorder, are characterized by sleep problems. Research indicates melatonin exerts a sleep-promoting action by accelerating sleep initiation, improving sleep maintenance, and marginally altering sleep architecture. Melatonin is also found to exhibit beneficial effects on chemotherapy-induced thrombocytopenia, because of its strong antioxidant action.

Melatonin undergoes first-pass hepatic metabolism and exhibits rapid absorption and short half-life (40-50 min), because of which, peak plasma concentrations are reached between 20 and 120 min after ingestion of an immediate-release formulation and decline within 1.5 h, depending on dose. As an effect of these pharmacokinetic parameters, high doses of melatonin would be required to maintain effective bodily concentrations throughout the night which lead to risk of receptor desensitization and unnecessary burden to liver function. Therefore, modified release formulation of melatonin would be desirable which circumvents the fast clearance of the hormone by releasing the hormone in the gut over an extended period, thereby mimicking physiological patterns of melatonin secretion and providing desired health benefits.

Literature indicates that researchers have worked on melatonin controlled or sustained release preparations for oral administration using different type of carriers.

U.S. Pat. No. 8,962,024 relates to a method of treating insomnia and improving restorative sleep quality in patients which involves administering the patients with composition comprising one compound selected from melatonin, other melatonergic agents, melatonin agonists and melatonin antagonists along with other excipients. US′024 also claims the prolonged release composition comprising melatonin prepared by direct compression method with ground powder mass of an acrylic resin along with directly compressible excipient

U.S. Pat. No. 6,833,383 describes treating patients with benzodiazepine dependency which involves administering melatonin in a controlled release form, wherein the melatonin is in particulate form comprising particles coated with a physiologically acceptable coating material wherein the coating material used is acrylic polymer.

U.S. Pat. No. 5,879,710 relates to a mucoadhesive controlled release dosage with a principle active agent as melatonin and melatonin derivative which comprises of two layers, wherein the first layer being mucoadhesive and permitting a sustained release of the active and the second layer being non-mucoadhesive. The polymer used in the first and second layer comprises of acrylic polymer and cellulose polymers for controlling release.

US patent application 20080181943 describes a composition comprising combination of a long-acting hypnotic agent and short acting hypnotic agent such as melatonin for treating sleep disorders. The composition comprises of multilayer tablet wherein comprising a sustained release short acting hypnotic. The sustained release layer may be prepared by incorporating a matrix system which comprises of polymers like hydroxypropylmethylcellulose, hydroxymethylcellulose and hydroxyethylcellulose or by coating immediate release tablet/layer with the polymers like ethyl cellulose copolymers and also methyl methacrylate polymers.

US patent application 20080171085A1 relates to a biphasic composition comprising first solid phase containing sustained-release matrix of hydroxymethyl cellulose for melatonin, and second water soluble phase including immediate release agent.

Korean patent application KR501827 describes multilayer sustained release system which comprises of an active layer including melatonin as an active and coating layer of acrylic acid copolymer, the methacrylic acid copolymer, the methacrylic acid/acrylic acid ethyl copolymer, the poly (ethyl acrylate/methyl methacrylate) ester.

Prior art references relate to melatonin formulations which make use of cellulose polymers and other hydrophilic swelling excipients to achieve controlled release from tablet and capsule formulations. However, release of melatonin from hydrophilic swelling polymeric system may not be reliable and reproducible due to possible dose dumping. Also use of cellulose polymers, such as ethyl cellulose, may result into incomplete release of melatonin from tablet or capsule dosage forms. There is a need to develop melatonin formulations, which are convenient to administer and would also ensure consistent release pattern of the active and should help the subject to maintain effective body levels of melatonin over the desired time duration.

### SUMMARY

It was surprisingly found that the inventors could achieve desired release profile of melatonin over a period of 6 to 10 hours, as described herein, through optimal selection of type and concentration of non-swelling release controlling agent as well as the optimized process for preparation. The researchers have come up with granular modified release melatonin compositions, which are smooth, slippery, exhibit taste masking and can be formulated in gummies and jellies, without imparting any gritty feeling in the mouth. No prior art references mention modified release melatonin gummy formulations till date. The granular composition can be also formulated in other desired oral dosage forms like chewing gums, and swallowable dosage forms like pellets, minitablets, tablets, caplets, capsules, beverages or can be filled in sachets for intended use by the subjects.

Melatonin granular composition for oral administration is comprised of at least one non-swelling release controlling agent in which melatonin is uniformly embedded, using controlled conditions of heat. The granular melatonin composition may be comprised of at least one non-polymeric non-swelling release controlling agent, optionally combined with polymeric non-swelling release controlling agent using suitable process of granulation and/or coating to get melatonin granules ranging in size from 50 to 600 microns. These smooth, uniform and taste masked granules are suitable for incorporating into the formulations like gummies and jellies, for convenient administration to elderly subjects of modified release of melatonin over a period of 6 to 10 hours. Melatonin compositions as described herein are easy to prepare, using commonly available equipment and ensure modified release of active over extended time duration. The formulations can be used to improve sleep quality in subjects experiencing disturbance in circadian rhythm due to deficiency in endogenous melatonin, jetlag conditions or subjects working in multiple shifts. The formulations can be also useful in subjects suffering from neuropsychiatric disorders, migraine, epilepsy, and cancer conditions, where sound sleep plays a critical role.

### Objectives

The main objective of the invention is to provide modified release melatonin composition for oral administration comprising at least one non-swelling release controlling agent.

One more objective of the invention is to provide melatonin matrix granular composition comprising of about 10 to 70% by weight of non-swelling release controlling agent and at least one excipient acceptable in nutraceutical, pharmaceutical and/or food industry.

One important objective of the invention is to provide modified release composition comprising about 1 to 70% of melatonin, at least 10 to 70% by weight of one or more non-swelling release controlling agent and at least one excipient. The granular melatonin composition may be comprised of at least one non-polymeric non-swelling release controlling agent, optionally combined with polymeric non-swelling release controlling agent.

Still one objective of the present invention is to provide melatonin modified release composition which exhibits about 80% release of melatonin over 6-10 hours.

According to one more objective of the present invention is to provide modified release melatonin composition comprising release controlling agents selected from fats, lipids, waxes, oils, fatty acids, fatty acid esters and the combination thereof.

One important objective of the invention is to provide a process for preparation, wherein melatonin is uniformly embedded using at least one non-polymeric non-swelling, release controlling agent using controlled heat conditions. The granules may be optionally treated with polymeric non-swelling release controlling agent by suitable process of granulation and/or coating to obtain modified release composition.

One more objective of the invention is to provide modified release composition, which may be optionally comprised of at least about 5% by weight of non-swelling polymeric release controlling agent.

Another objective of the invention is to provide a modified release melatonin composition comprising matrix granules ranging from particle size 50 to 600 microns, which are uniform, taste masked and can be formulated in dosage forms like gummies, jellies and chewing gums, which are convenient for administration without causing any grittiness in the mouth.

One more objective of the invention is to provide a modified release melatonin composition in granular form, which can be filled in sachets or it can be formulated into pellets, minitablets, tablets, capsules, beverages for intended use by the subjects.

### DETAILED DESCRIPTION

The invention relates to a modified release composition of melatonin for oral administration. The composition is comprised of melatonin uniformly dispersed and embedded in a matrix of at least one non-swelling release controlling agent and at least one excipient, which is acceptable in nutraceutical, pharmaceutical and/or food industry. The matrix granular composition may be optionally further comprised of polymeric non-swelling release controlling agent. The composition may be comprised of about 1 to 70% by weight of melatonin and about 10 to 70% by weight of at least one non-swelling release controlling agent. The invention also provides process for preparation of melatonin modified release composition in the form of granules, which can be conveniently formulated in desired dosage form such as gummies and jellies intended for oral administration of modified release formulation to the subjects in need thereof. The composition may release about 80% of melatonin over a period of 6 to 10 hours. The granular composition can also be formulated in other suitable oral dosage forms such as pellets, minitablets, tablets, capsules, beverages or can be filled in sachets.

The term ‘modified release’ as used herein refers to the release pattern of the active from melatonin granular composition, at desired rate over extended time. The composition, as described herein, is designed in such a way that the active would be released slowly over prolonged time as per the requirement, to suit its intended use of improving sleep quality in the subjects. Accordingly, the active would be released in the body for providing effective amount, to induce sleep in initial phase of 1 hour. The composition would release the active in modified way, in order to maintain desired amount of active in the body of the subject, over extended hours of sleep as per requirement. Modified release melatonin composition, as described herein makes use of matrix of non-swelling release controlling agent, which avoids dose dumping in initial phase as well as incomplete drug release in later phase, thus making the active available at a constant and pre-determined rate during entire sleep duration.

The term ‘composition’ or ‘formulation’ may be used interchangeably within the scope of the present invention, wherein it relates to melatonin granules comprising the active, release controlling agent and the excipient. The composition or formulation also refers to the final dosage form, according to which the granules may be used as such, by filling in sachets or can be converted in suitable dosage forms for oral administration, such as gummies, jellies, chewing gums, capsules or other compressible and swallowable forms including tablets, pellets, minitablets and the like, which are generally known to the person skilled in the art.

The term ‘subject in need thereof’ as used herein refers to the subjects of all age groups, including paediatric and geriatric category as well, who are suffering from poor quality of sleep due to sleep disorders or disturbed sleep cycle, thus require the support of the supplements or aids, to improve quality of sleep. The subjects in need thereof, may be suffering from insomnia condition, or disturbed circadian rhythm due to jetlag or due to working in day-night shifts and also because of anxiety experienced before and after surgery. The subjects in need thereof may be suffering from ailments like neuropsychiatric disorders, migraine, epilepsy or cancer, wherein the sound sleep is very important for recovery process. The composition comprising modified release melatonin may be useful to improve sleep quality in all the subjects who are in need of such improvement, due to variety of reasons.

The term ‘non-swelling release controlling agent’ as used herein refers to the carrier or the component employed in the composition for preparation of uniform and smooth granules of melatonin and also for achieving the characteristic desired release profile of melatonin. These release controlling agents are hydrophobic in nature, insoluble in water and do not swell in contact with the aqueous phase. These non-swelling release controlling agents also act as continuous phase, in which melatonin is uniformly distributed by suitable process to get matrix embedded granules. These components contribute for getting smooth, uniform and taste masked granules of melatonin, which provide modified release of melatonin over a period of 6 to 10 hours. The non-polymeric release controlling agents may be preferably obtained from natural source, although the agents may be employed from synthetic and semi-synthetic sources.

As per one embodiment of the invention, the non-swelling release controlling agent, as described herein may be selected from non-polymeric and/or polymeric release controlling agents, and the combination thereof. The granular composition is preferably comprised of non-swelling, non-polymeric release controlling agents. The composition may also further optionally be comprised of polymeric release controlling agents.

As per one important embodiment, the non-swelling, non-polymeric release controlling agent may be selected from the class of, but not limited to lipids, fats, waxes and the combination thereof. The non-swelling release controlling agent may be selected from, but not limited to, saturated fatty acids having 12 to 28 carbons, such as stearic acid, fatty alcohols having from 16 to 44 carbons, cetyl alcohol, pegylated fatty acids, glycerol fatty acid esters, monoglycerides, diglycerides, triglycerides, derivatives of mono-diglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, polyglycerol esters, glycerolipid, phosphatic acid, phosphatidylethanolamine, phosphatidylcholine, phosphatidylserine, phosphatidylinositol or other glycerophospholipids, ceramide, sphingolipid, sterol, fat-soluble vitamin, prenol, saccharolipid, polyketide, their salts and esters and the combination thereof. The release controlling agent may also be selected from, but not limited to, beeswax, candelilla wax, carnauba wax, spermaceti, paraffin wax, synthetic waxes and the combination thereof.

As per one more embodiment of this invention, the composition may optionally be comprised of polymeric non-swelling release controlling agent selected from the class of, but not limited to shellac; cellulose derivatives such as cellulose acetate phthalate (CAP), cellulose acetate trimellitate (CAT), cellulose acetate, cellulose acetate butyrate, ethyl cellulose; lactic acid copolymers such as polylactic acid, polylactic-co-glycolic acid, vinyl polymers and derivatives such as polyvinylpyrrolidone, polyvinyl chloride, polyvinyl alcohol, polyvinyl acetate, mixture of polyvinyl acetate and polyvinylpyrrolidone (Kollidon SR), polymethacrylic acid and derivatives such as poly(methacrylic acid-co-methyl methacrylate), polyacrylamide, polyethylene oxide and the derivatives, and the combination thereof.

According to one more embodiment, melatonin composition may contain non-swelling release controlling agent along with at least one nutraceutically and/or pharmaceutically acceptable excipient. The composition may preferably be comprised of one or more non-polymeric non-swelling release controlling agent/s along with at least one excipient, acceptable in nutraceutical, pharmaceutical and food industry. Further matrix embedded melatonin granules composition may optionally be comprised of at least 5% by weight of polymeric non-swelling release controlling agent, by process of granulation and/or coating to get taste masked, uniform and smooth granules.

The term ‘melatonin’ as used herein refers to the active employed in the present invention, which is available as white to off-white, crystalline powder. It may be obtained from natural or synthetic source. Melatonin is a hormone that is produced in the brain in response to darkness which helps with the timing of circadian rhythms and with sleep. Melatonin can also be a therapeutic chemically synthesized form of the pineal indole melatonin with antioxidant properties. Melatonin is slightly soluble in water, soluble in acetone, ethyl acetate and methanol.

As per one important embodiment of the invention, the composition, as described herein may be comprised of about 1 to 70% by weight of melatonin, uniformly distributed in about 10 to 70% by weight of at least one non-swelling release controlling agent and at least one excipient acceptable in nutraceutical, pharmaceutical and food industry.

According to one embodiment, the composition may be preferably comprised of about 5 to 65% by weight of melatonin. Most preferably the composition may be comprised of about 10 to 60% by weight of melatonin, which is dispersed in the non-swelling release controlling agent and at least one excipient acceptable in nutraceutical, pharmaceutical and food industry.

As per important embodiment, the composition may be advantageously comprised of about 15 to 65% by weight of at least one non-swelling release controlling agent, in which melatonin is dispersed well to form the matrix, which may be optionally treated with polymeric non-swelling release controlling agent

As per one embodiment of the invention, modified release composition may be comprised of at least one excipient, which may facilitate the granulation of melatonin. The excipient may also help as a processing aid in formulating the granules in desired dosage form intended for oral administration. These are commonly used ingredient in nutraceutical, pharmaceutical and food industry, and may be selected from the group of, but not limited to, fillers, diluents, disintegrants, lubricants, binders, glidants, anti-caking agents, stabilizers, surfactants, channelizing agents, vehicles, buffers, stabilizers, preservatives, acidifiers, alkalizers, complexing agents, gum bases, antioxidants, viscosity enhancers, gelling agents, plasticizers, coating materials, sweeteners, colours, flavours and the combination thereof.

Modified release composition of melatonin as described herein, may be comprised of about 10 to 60% by weight of at least one excipient, which is selected from natural, semi-synthetic or synthetic sources.

The formulation may be comprised of diluents known in the art, but not limited to microcrystalline cellulose, silicified microcrystalline, powdered cellulose, microfine cellulose, corn starch, rice bran extract, mannitol, maltodextrin, calcium phosphate, dibasic calcium phosphate, tribasic calcium phosphate, calcium sulfate, or mixtures thereof. The diluents may also be selected from glucose, lactose, sucrose, dextrose, fructose, compressible sugar, or mixtures thereof.

The binders may be selected from the group of low viscosity cellulose derivatives such as hydroxypropylmethyl cellulose (HPMC), hydroxypropyl cellulose (HPC), ethylcellulose, carboxymethylcellulose (CMC), sodium CMC, potassium CMC, calcium CMC, methylcellulose, hydroxyethyl cellulose (HEC), microcrystalline cellulose; polyvinylpyrrolidone (PVP), vinyl pyrrolidone-vinyl acetate copolymer, polyvinyl alcohol, starch, gums like xanthan gum, guar gum, acacia, locust bean gum, gellan gum, alginates, or mixtures thereof.

The disintegrants may be selected from sodium starch glycolate, crospovidone, croscarmellose sodium, croscarmellose calcium, croscarmellose potassium, sodium carbonate, sodium hydrogen carbonate, calcium carbonate, starch, starch 1500, modified starch, pregelatinized starch, crosslinked carboxymethyl starch, sodium hydrogen carbonate, hydroxypropyl cellulose, sodium carboxymethylcellulose or mixtures thereof.

The lubricants may be selected from magnesium stearate, calcium stearate, sodium benzoate, talc, or mixtures thereof.

The glidants may be selected from suitable glidants known in the art and commonly used in the industry, selected from the group of stearate, starch, talc and the derivatives.

According to the important embodiment of the invention, the process for preparation of melatonin composition may be comprised of mixing melatonin with at least one non-swelling release controlling agent and at least one excipient which is acceptable in nutraceutical, pharmaceutical or food industry. The blend is subjected to suitable process of granulation such as slugging, dry granulation, direct compression, extrusion spheronization, compaction, compression, melt granulation, melt extrusion, melt solidification, spray-drying, freeze-drying and spray chilling.

The matrix granules obtained in this step may optionally be treated with polymeric non-swelling release controlling agent using suitable process of granulation and/or coating such as wet granulation, dry granulation, spray coating, fluidized bead coating, pan coating and the like, to obtain uniform, smooth and taste masked granules.

As per one more embodiment of the invention, the process for preparation of melatonin formulation employs commonly available and easy to use industrial equipment. Suitable parameters of temperature, revolutions and torque may be selected for carrying out melt granulation. The process may be carried out by varying the temperature in the range of 20 to 100° C. and the molten form can be processed to get matrix granules of desired size. The granules obtained in this process can be optionally treated with polymeric non-swelling release controlling agent, employing suitable process of granulation and/or coating. The solvent employed for granulation and/or coating may be aqueous, organic or the combination thereof. The composition of melatonin as described herein is granular in nature, the granules being uniform, smooth and taste masked, which can be conveniently converted in desired dosage form suitable for oral administration gummies, jellies or other suitable dosage forms such as tablets, capsules, caplets, pellets, minitablets and the like. The process for preparation is easy, economic and also makes use of commonly available industrial equipment.

According to one embodiment of the invention, size of the granules may vary from 50 micron to 600 microns. Melatonin granules can be used as such in stick packs, can be mixed in beverages, filled in the capsules or compressed in the tablets to get final dosage form for oral administration. The composition also exhibits effect of taste masked granules. The granules are smooth, uniform and do not exhibit any grittiness in the mouth and thus can be formulated in solid oral dosage forms like gummies, jellies, candies and chewing gums, for easy and convenient administration to geriatric and paediatric subjects, as per the need, for providing the intended effect. The formulations of the present invention can be in the form of granules filled in sachets and stick packs, tablets, capsules, caplets, pills, beads, beadlets, pellets, beverages and the like.

The modified release formulation, as described herein is evaluated for stability in granular form as well as finished dosage form. The formulation is also analysed for drug release as well as percentage assay content and the granules can be converted in convenient dosage form for administration to the subjects in need thereof, for improvement in sleep quality.

The granular composition or solid finished dosage formulation as described herein, is evaluated for dissolution profile using USP Type II (paddle) apparatus at 100 rpm speed, using 900 ml of 0.1 N HCL as dissolution medium at temperature 37° C.±Amount of melatonin released from the formulation is measured by using HPLC method using Potassium Dihydrogen Orthophosphate Buffer pH 3.5:Acetonitrile (75:25) as the mobile phase.

The modified release composition as described herein provides reliable, reproducible and consistent release of about 80 to 95% of melatonin over the duration of 6 to 10 hours. The modified release composition of the present invention may release 30 to 60% of melatonin in first hour, followed by about 55 to 70% of release in 3 to 6 hours, and about 70 to 95% of melatonin in 6-10 hours. Thus, the active would be released in the body system for providing effective amount, to induce sleep in initial phase of 1 hour. The composition would release the active in modified way, to maintain desired amount of active in the body of the subject, over extended hours as per requirement.

The modified release compositions, as described herein, provides slow release of melatonin over a longer duration and it can be useful to improve sleep quality in subjects suffering from insomnia condition, or those who are suffering from disturbed circadian rhythm due to jetlag or due to working in day-night shifts and also in the subjects who are suffering from anxiety before and after surgery. It may be also useful in children having sleep related disorders and, in the subjects, suffering from delayed sleep-wake phase cycle. The composition may be also useful as a hypnotic or sedative agent in subjects suffering from ailments like neuropsychiatric disorders, migraine, epilepsy or cancer, wherein the sound sleep is very important for recovery of the subjects.

The composition can be formulated in swallowable formulations such as tablets, caplets, capsules or the non-swallowable buccal formulations like gummies, candies or chewing gums, which makes it more convenient for children and elderly subjects, thus offering the advantage of enhanced compliance.

The invention is now illustrated with non-limiting examples.

### EXAMPLES

### Example 1: Composition of Melatonin Modified Release Granules

| | | |
|---|---|---|
| Sr. no | Ingredients | Percentage % |

Granulation I

| | | |
|---|---|---|
| 1 | Melatonin | 47.17 |
| 2 | Mannitol | 18.87 |
| 3 | Stearic acid | 9.43 |
| 4 | Carnauba Wax | 18.87 |

Granulation II

| | | |
|---|---|---|
| 5 | Polyvinyl acetate 30% | 5.66 |
| | Dispersion | |
| 6 | Purified Water | qa |
| | | 100.00 |

### Process for Preparation

Melatonin (47.17%), mannitol (18.87%), stearic acid (9.43%), carnauba wax (18.87%) mixture was blended well and sifted. The mixture was fed into a hot melt granulator, while maintaining the feed temperature and torque. The temperature was varied from 25-75° C. and the torque was at 30-40%. The granules were collected and further treated with 30% polyvinyl acetate dispersion. The collected granules were dried at 45° C. and sieved to get the size in the range of 75-250 microns.

The formulation was evaluated for dissolution profile using USP Type II (paddle) apparatus at 100 rpm speed, using 900 ml of 0.1 N HCL as dissolution medium at temperature 37° C.±0.5° C. Melatonin released from the formulation is measured by using HPLC method using Potassium Dihydrogen Orthophosphate Buffer pH 3.5:Acetonitrile (75:25) as the mobile phase. The dissolution data is provided in Table 1.

TABLE 1
Dissolution profile of composition of Example 1

| Time | Cumulative % Melatonin Release |
|---|---|
| 1 hour | 49 |
| 3 hours | 63 |
| 6 hours | 68 |

Melatonin formulation exhibits modified release pattern wherein 49% of active is released in the first hour and 63-68% drug is released within 6 hrs.

### Example 2: Composition of Melatonin Modified Release Granules

| Sr. no | Ingredients | Percentage % |
|---|---|---|
| 1 | Melatonin | 31.25 |
| 2 | Mannitol | 37.50 |
| 3 | Stearic acid | 6.25 |
| 4 | Carnauba Wax | 25.00 |
| | | 100.00 |

### Process for Preparation

Melatonin (31.25%), mannitol (37.50%), stearic acid (6.25%) and carnauba wax (25%) were blended well and sifted. The mixture was fed into a hot melt granulator, while maintaining the feed temperature and torque. The temperature was varied from 25-75° C. and the torque was maintained at 30-40%. The granules obtained were sifted and dried. The granules thus obtained were in the size range of 150-250 microns.

The formulation was evaluated for dissolution profile using USP Type II (paddle) apparatus at 100 rpm speed, using 900 ml of 0.1 N HCL as dissolution medium at temperature 37° C.±0.5° C. Melatonin released from the formulation is measured by using HPLC method mobile phase Potassium Dihydrogen Orthophosphate Buffer pH 3.5:Acetonitrile (75:25). The dissolution data is provided in Table no. 2

TABLE 2
Dissolution profile of composition
of Example 2

| | Cumulative % |
|---|---|
| | Melatonin |
| Time | Release |
| 1 hour | 33 |
| 3 hours | 65 |
| 6 hours | 82 |

The dissolution profile of melatonin formulation exhibits modified release pattern wherein 33% is released in the first hour and 65-82% drug is released within 3-6 hrs.

### Example 3: Composition of Melatonin Modified Release Granules

| Sr. no | Ingredients | Percentage % |
|---|---|---|
| 1 | Melatonin | 25.00 |
| 2 | Mannitol | 50.00 |
| 3 | Stearic acid | 5.00 |
| 4 | Carnauba Wax | 20.00 |
| | | 100.00 |

### Process for Preparation

The process as described in Example 2 was followed to prepare melatonin compositions. The melt granulated mass was sifted to get granules in the range of 150 to 250 micron.

The release profile was checked using dissolution apparatus, USP Type II (paddle) apparatus at 100 rpm speed, using 900 ml of 0.1 N HCL as dissolution medium at temperature 37° C.±0.5° C. Melatonin released from the formulation is measured by using HPLC method using mobile phase Potassium Dihydrogen Orthophosphate Buffer pH 3.5:Acetonitrile (75:25). The dissolution data is provided in Table 3.

TABLE 3
Dissolution profile of composition
of Example 3

| | Cumulative % |
|---|---|
| | Melatonin |
| Time | Release |
| 1 hour | 34 |
| 3 hours | 65 |
| 6 hours | 85 |

The dissolution profile of melatonin formulation exhibits modified release pattern wherein 34% is released in the first hour and 65-85% drug is released within 3-6 hrs.

### Example 4: Composition of Modified Release Granules

| Sr. no | Ingredients | Percentage % |
|---|---|---|
| 1 | Melatonin | 50.00 |
| 2 | Mannitol | 20.00 |
| 3 | Stearic acid | 10.00 |
| 4 | Carnauba Wax | 20.00 |
| | | 100.00 |

### Process for Preparation

Same process, as described in Example 2 was followed to prepare melatonin formulations. The granules form was collected and processed to get granules in the range of 75 to 250 micron.

The release profile was checked using dissolution apparatus, USP Type II (paddle) apparatus at 100 rpm speed, using 900 ml of 0.1 N HCL as dissolution medium at temperature 37° C.±0.5° C. Melatonin released from the formulation is measured by using HPLC method using mobile phase Potassium Dihydrogen Orthophosphate Buffer pH 3.5:Acetonitrile (75:25) as the mobile phase. The dissolution data is provided in Table 4.

TABLE 4
Dissolution profile of composition
of Example 4

| | Cumulative % |
|---|---|
| | Melatonin |
| Time | Release |
| 1 hour | 33 |
| 3 hours | 60 |
| 6 hours | 71 |

The dissolution profile of melatonin formulation exhibits modified release pattern wherein 34% is released in the first hour and 60-71% drug is released within 3-6 hrs.

### Example 5: Composition of Modified Release Granules

| | | |
|---|---|---|
| Sr. no | Ingredients | Percentage % |

Granulation I

| | | |
|---|---|---|
| 1 | Melatonin | 47.17 |
| 2 | Mannitol | 18.87 |
| 3 | Stearic acid | 9.43 |
| 4 | Carnauba Wax | 18.87 |

Granulation II

| | | |
|---|---|---|
| 5 | Methacrylate Copolymer 30% | 5.66 |
| 6 | Purified Water | sb |
| | | 100.00 |

### Process for Preparation

Melatonin (47.17%), mannitol (18.87%), stearic acid (9.43%), carnuba wax (18.87%) mixture was blended well. The mixture was fed into a hot melt granulator, while maintaining the feed temperature and torque. The temperature was varied from 25-75° C. and the torque was at 30-40%. The granules were collected and sifted. The granules were further treated with 30% methacrylate copolymer 30% dispersion. The collected granules were dried and further sieved to get the size in the range of 75-250 microns. The formulation was evaluated for dissolution profile using USP Type II (paddle) apparatus and the active released is measured by using HPLC method. The dissolution data is provided in Table 5.

TABLE 5
Dissolution profile of composition
of Example 5

| | Cumulative % |
|---|---|
| | Melatonin |
| Time | Release |
| 1 hour | 51 |
| 3 hours | 71 |
| 6 hours | 81 |

The dissolution profile of melatonin formulation exhibits modified release pattern wherein 51% is released in the first hour and 71-81% drug is released within 3-6 hrs.

### Example 5A: Comparative Dissolution Study in Dissolution Media of Varying pH

The formulation of Example 5 was subjected to comparative dissolution study in varying pH conditions. The dissolution study was performed using 900 ml dissolution media of each pH 6.2, pH 6.8 and pH 7.4 using USP Type II (paddle) apparatus at 100 rpm speed, at temperature 37° C.±0.5° C. Melatonin released from the formulation was measured by using HPLC method using Potassium Dihydrogen Orthophosphate Buffer pH 3.5:Acetonitrile (75:25) as the mobile phase. The dissolution data is provided in Table 6.

TABLE 6
Dissolution profile of Example 5 at varying pH buffer solution

| Dissolution | Percentage release of melatonin | | | | |
|---|---|---|---|---|---|
| Media | 1^{st} hour | 2^{nd} hour | 4^{th} hour | 6^{th} hour | 8^{th} hour |
| pH 6.2 | 45 | 56 | 67 | 73 | 75 |
| pH 6.8 | 50 | 60 | 69 | 74 | 77 |
| pH 7.4 | 52 | 63 | 73 | 80 | 85 |

In-vitro dissolution profile of the Formulation 5 in varying pH conditions indicate that release of melatonin from the composition is substantially similar in all media, which is about 45 to 52% in first hour and 75 to 85% at the end of eight hour. Thus the release profile from the formulation of the invention is independent of pH of the dissolution medium.

### Example 5B: Stability Study of Formulation 5

The formulation was packed in LDPE bags followed by aluminium bags in HDPE container and were stored at 40° C./75% RH and 30° C./75% RH for 3 months and 6 months respectively to analyse the stability of the formulation. The formulation was evaluated for dissolution studies using USP Type II (paddle) apparatus at 100 rpm speed, using 900 ml of 0.1N HCL and percent assay was also determined at the interval of 3 and 6 months.

Melatonin released from the formulation is measured by using HPLC method using Potassium Dihydrogen Orthophosphate Buffer pH 3.5:Acetonitrile (75:25) as the mobile phase.

TABLE 7
Stability Study of Formulation 5

| | | | | | | Assay |
|---|---|---|---|---|---|---|
| | | Percentage release in 0.1N HCL | | | | value |
| Condition | Duration | 1^{st} hour | 4^{th} hour | 6^{th} hour | 8^{th} hour | % w/w |
| | Initial | 49 | 73 | 77 | 81 | 100.7 |
| 40° C./ | 3 | 48 | 74 | 78 | 81 | 99.9 |
| 75% RH | months | | | | | |
| | 6 | 49 | 75 | 78 | 82 | 99.8 |
| | months | | | | | |
| 30° C./ | 3 | 49 | 76 | 79 | 83 | 98.7 |
| 75% RH | months | | | | | |
| | 6 | 45 | 74 | 80 | 82 | 98.6 |
| | months | | | | | |

The dissolution profile as well as assay value of the formulation as per Table 7 indicates that the formulation is stable over 6 months at both storage conditions of temperature and relative humidity.

### Example 6: Composition of Melatonin Modified Release Granules

| | | |
|---|---|---|
| | | Quantity |
| | | (% w/w) |
| Sr. No. | Ingredients | Formula |

Granulation I

| | | |
|---|---|---|
| 1 | Melatonin | 48.54 |
| 2 | Mannitol | 19.42 |
| 3 | Stearic acid | 9.71 |
| 4 | Carnauba Wax | 19.42 |

Granulation II

| | | |
|---|---|---|
| 5 | Polyvinyl acetate 30% | 2.91 |
| 6 | Purified Water | q.s |

| | |
|---|---|
| Total | 100.00 |
| Particle Size | 75 to 250 microns |

### Process of Preparation

The process as described in Example 1 was followed to prepare melatonin composition. The granules were collected after completing granulation I phase and further treated with 30% polyvinyl acetate dispersion. The collected granules were dried at 45° C. and sieved to get the size in the range of 75-250 microns.

The release profile was checked using dissolution apparatus, USP Type II (paddle) apparatus at 100 rpm speed, using 900 ml of 0.1 N HCL as dissolution medium at temperature 37° C.±0.5° C. Melatonin released from the formulation was measured by using HPLC method using mobile phase Potassium Dihydrogen Orthophosphate Buffer pH 3.5:Acetonitrile (75:25).

The dissolution data is provided in Table 8.

TABLE 8
Dissolution profile of composition
of Example 6

| | Cumulative % |
|---|---|
| | Melatonin |
| Time | Release |
| 1 hour | 49 |
| 3 hours | 63 |
| 6 hours | 73 |

The cumulative release profile indicates that melatonin formulation of Example 6 exhibits modified release pattern wherein 49% of melatonin is released in the first hour and 73% of melatonin is released at the end of 6 hours.

### Example 7-11: Composition of Modified Release Granules

| | EXAMPLE 7 | EXAMPLE 8 | EXAMPLE 9 | EXAMPLE 10 | EXAMPLE 11 |
|---|---|---|---|---|---|
| Ingredients | Percentage | Percentage | Percentage | Percentage | Percentage |
| Melatonin | 30.00 | 20.00 | 50.00 | 50.00 | 70.00 |
| Cetyl alcohol | 70.00 | — | — | — | — |
| Glyceryl | — | 80.00 | — | — | — |
| Monostearate | | | | | |
| Glyceryl | — | — | — | 50.00 | — |
| Behenate | | | | | |
| Hydrogenated | — | — | 30.00 | — | — |
| Castor oil | | | | | |
| Carnauba Wax | — | — | — | — | 20.00 |
| Mannitol | — | — | 20.00 | — | — |
| Dicalcium | — | — | — | — | 10.00 |
| Phosphate | | | | | |
| Total | 100 | 100 | 100 | 100 | 100 |
| Particle Size | 250 to 400 | 300 to 600 | 250 to 400 | 75 to 250 | 75 to 250 |
| | microns | microns | microns | microns | microns |

### Process of Preparation

The process as described in Example 2 was followed to prepare melatonin compositions. The melt granulated mass was sifted to get granules in the desired particle size.

The release profile was checked using dissolution apparatus, USP Type II (paddle) apparatus at 100 rpm speed, using 900 ml of 0.1 N HCL as dissolution medium at temperature 37° C.±0.5° C. Melatonin released from the formulation is measured by using HPLC method using mobile phase Potassium Dihydrogen Orthophosphate Buffer pH 3.5:Acetonitrile (75:25). The dissolution data is provided in Table 9.

TABLE 9
Dissolution profile of composition of Example 7-11

| Time | | | | | |
|---|---|---|---|---|---|
| (hr.) | Cumulative % Melatonin Release | | | | |
| 1 | 46 | 52 | 47 | 53 | 35 |
| 4 | 63 | 76 | 79 | 77 | 63 |
| 8 | 76 | 87 | 90 | 88 | 78 |

The cumulative release profile indicates that melatonin formulations of Example 7 to 11 exhibit modified release pattern wherein 35 to 46% of melatonin is released in the first hour, 63 to 79% of melatonin is released in 4 hours and 76 to 90% of melatonin is released at the end of 8 hours.

### Example 12: Formulation of Gummies Using Melatonin Modified Release Granules of Example 5

200 gm sucrose was dissolved in sufficient amount of distilled water and this mixture was heated to boiling, followed by addition of glucose syrup to the hot viscous solution with stirring.

A 100 ml solution of (0.5 g) sodium citrate and (4.5 g) citric acid was added to step a and the resulting mixture was stirred well and filtered.

Carrageenan (10 g) and Gellan Gum (40 g) were dispersed in hot distilled water and stirred to obtain a homogeneous system. Viscous solution obtained from step a was added to this system, followed by additives such as strawberry flavour (2 g) and finally melatonin granules obtained from example 5 were added.

The prepared mass was blended well and poured into the silicon mould which was coated with 1 g carnauba wax. The mould was left to set at room temperature to dry for 24 to 48 h.

Each 2 g of gummy contains 0.5% of Melatonin i.e., 10 mg of Melatonin.

The release profile of gummies was checked using dissolution apparatus, USP Type II (paddle) apparatus at 100 rpm speed, using 900 ml of 0.1 N HCL as dissolution medium at temperature 37° C.±0.5° C.

Melatonin released from the formulation is measured by using HPLC method using mobile phase Potassium Dihydrogen Orthophosphate Buffer pH 3.5:Acetonitrile (75:25). The dissolution data is provided in Table no 10.

The formulation was also evaluated for its stability, wherein the formulation was stored 40° C./75% RH for 3 months and analysed based on dissolution profile in comparison with initial sample of gummies.

TABLE 10
Dissolution profile of gummy composition of Example 12

| | | Percentage release in 0.1N HCL | | | |
|---|---|---|---|---|---|
| Condition | Duration | 1^{st} hour | 4^{th} hour | 6^{th} hour | 8^{th} hour |
| | Initial | 41 | 79 | 93 | 98 |
| 40° C./75% RH | 3 months | 38 | 73 | 88 | 92 |

### Example 13: Capsule Formulation Using Melatonin Modified Release Granules of Example 7

Modified-release formulation of Example 7 equivalent to 10 mg melatonin was mixed with microcrystalline cellulose and dicalcium phosphate (1:1) and lubricated with 2% silicon dioxide. The mixture was filled in capsules and subjected to dissolution studies.

TABLE 11
Dissolution profile of composition of Example 13

| | Percentage release in 0.1N HCL media | | |
|---|---|---|---|
| Dosage Form | 1^{st} hour | 4^{th} hour | 8^{th} hour |
| Capsule | 53 | 67 | 74 |

The dissolution study of capsule of Example 13 (Table 11) indicates that the formulation exhibits modified release of melatonin, wherein 74% of the active is released in 8 hours.

### Example 14: Capsule Formulation Using Modified Release Granules of Example 10

Granules of modified-release composition of Example 10, equivalent to 10 mg melatonin was mixed with microcrystalline cellulose and dicalcium phosphate. This mixture was lubricated with 2% silicon dioxide. 250 mg of this mixture was filled in capsules and subjected to dissolution studies.

TABLE 12
Dissolution profile of Capsule formulation of Example 14

| | Percentage release in 900mL 0.1N HCL media | | |
|---|---|---|---|
| Dosage Form | 1^{st} hour | 4^{th} hour | 8^{th} hour |
| Capsule | 50 | 76 | 85 |

The dissolution profile indicates that capsule formulation of Example 14 exhibits modified release of melatonin over a period of 8 hours.

Modified release melatonin composition, as described herein makes use of matrix of at least one non-swelling release controlling agent and one excipient, which is acceptable in nutraceutical, pharmaceutical and food industry. The granules are stable, smooth and taste masked, ranging in size from 50 to 600 microns, which can be conveniently formulated in gummies, jellies, chewing gums, capsules or other compressible dosage forms. The modified release composition of the present invention may release 30 to 60% of melatonin in first hour, followed by about 55 to 70% of release in 3 to 6 hours, and about 70 to 95% of melatonin in 6-10 hours, thus making the active available at a constant and pre-determined rate. The formulation, as described herein may be useful for improvement in quality of sleep to the subjects in need thereof.

1. A modified release melatonin composition, comprising
1 to 70% of melatonin,
to 70% of at least one non-swelling release controlling agent, and
at least one excipient, which is acceptable in nutraceutical, pharmaceutical and food industry.

2. Melatonin composition of claim 1, which may be comprised of at least one non-swelling release controlling agent, selected from the group of non-polymeric, polymeric release controlling agent and the combination thereof.

3. Melatonin composition of claim 1, wherein non-polymeric non-swelling release controlling agent may be selected from the group of fats, lipids, waxes, oils, fatty acids, fatty acid esters and the combination thereof.

4. Melatonin composition of claim 3, wherein non-polymeric non-swelling release controlling agent may be selected from carnauba wax, beeswax, candelilla wax, paraffin wax, synthetic waxes, spermaceti, stearic acid, cetyl alcohol, pegylated fatty acids, monoglycerides, diglycerides, triglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, phosphatides, cerebrosides, gangliosides, cephalins, glycolipids, sulfatides, sugar esters, sugar ethers, sucrose esters, sterols, polyglycerol esters, glycerolipid, phosphatic acid, phosphatidylethanolamine, phosphatidylcholine, phosphatidylserine, phosphatidylinositol or other glycerophospholipids, ceramide, sphingolipid, sterol, fat-soluble vitamins, prenol, saccharolipid, polyketide, their salts and esters and the combination thereof.

5. Melatonin composition of claim 2, which may be optionally comprised of about 5% by weight of polymeric non-swelling release controlling agent.

6. Melatonin composition of claim 5, wherein polymeric non-swelling release controlling agent may be selected from shellac; cellulose derivatives such as ethyl cellulose, cellulose acetate phthalate (CAP), cellulose acetate trimellitate (CAT), cellulose acetate, cellulose acetate butyrate; lactic acid copolymers such as polylactic acid, polylactic-co-glycolic acid, vinyl polymers and derivatives such as polyvinylpyrrolidone, polyvinyl chloride, polyvinyl alcohol, polyvinyl acetate, mixture of polyvinyl acetate and polyvinylpyrrolidone, polymethacrylic acid and derivatives such as poly(methacrylic acid-co-methyl methacrylate), polyacrylamide, polyethylene oxide and the derivatives, and the combination thereof.

7. Melatonin composition of claim 1, wherein the excipient may be used in the range of 10 to 60% by weight of the composition.

8. Melatonin composition of claim 7, wherein the excipient may be selected from the group of fillers, diluents, disintegrants, lubricants, binders, glidants, anti-caking agents, stabilizers, surfactants, channelizing agents, vehicles, buffers, stabilizers, preservatives, acidifiers, alkalizers, complexing agents, gum bases, antioxidants, viscosity enhancers, plasticizers, coating materials, sweeteners, colors, flavors and the combination thereof.

9. A process for preparation of melatonin composition, which is comprised of
mixing melatonin with at least one non-swelling release controlling agent using controlled heat conditions in the range of 20 to 100° C. to get matrix granules of desired size;
optionally treating the granules with polymeric non-swelling release controlling agent, employing suitable process of granulation to obtain uniform, smooth and taste masked granules;
formulating the granules in final dosage form.

10. Melatonin composition of claim 1, which exhibits a modified release wherein about 80% of melatonin is released over 6 to 10 hours.

11. Process for preparation of melatonin composition of claim 9, wherein the granules range in size from 50 to 600 microns and can be formulated in suitable oral dosage forms like gummies, jellies, chewing gums, pellets, minitablets, tablets, capsules, beverages or can be filled in sachets and stick packs for improvement in quality of sleep in the subjects in need thereof.
